(19) United States
(12) Patent Application Publication
(10) Pub. No.: US 2025/0169501 A1
(43) Pub. Date: May 29, 2025

(54) Metal(loid)-Based Compositions and Uses Thereof Against Bacterial Biofilms

(71) Applicant: CCREST Laboratories Inc., Montreal (CA)

(72) Inventors: Raymond J. Turner, Calgary (CA); Ali Pormohammad, Calgary (CA)

(73) Assignee: CCREST Laboratories Inc., Montreal, QC (CA)

(21) Appl. No.: 18/840,794

(22) PCT Filed: Feb. 22, 2023
(86) PCT No.: PCT/IB2023/051621

§ 371 Date: Aug. 22, 2024

Related U.S. Application Data
(60) Provisional application No. 63/312,861, filed on Feb. 23, 2022.

(51) Int. Cl. A01N 59/16; A01P 1/00

(52) U.S. Cl. CPC A01N 59/16; A01P 1/00

(57) ABSTRACT

Described herein are new compositions comprising a combination of metal(loid)-based antimicrobials (MBAs) that are effective against bacterial biofilms. In embodiments the composition comprises a combination of metal(loid) selected from: a) at least two metals; and b) at least one metal and at least one metalloid. Examples of metal(s) and metalloid(s) useful in the combination include, silver nitrate, potassium tellurite, hydrogen tetrachloroaurate (III), nickel sulfate, copper (II) sulfate, aluminium sulfate, gallium nitrate, sodium selenite, and zinc sulfate. Some combinations are broad-spectrum while others are especially effective against bacterial biofilms resulting from the growth of Gram-negative bacteria or the growth of Gram-positive bacteria. The compositions may be used in methods for disruption and/or of removal of biofilm present on a surface, or for coating purposes thereby providing anti-biofilm activity to a surface.

### CROSS REFERENCE TO RELATED APPLICATION

This patent application claims priority to provisional patent application U.S. 63/312,861 filed on Feb. 23, 2022, the content of which is incorporated herein by reference in its entirety.

### FIELD OF THE INVENTION

The invention relates to the field of microbiology, and more particularly to compositions comprising a combination of metal(loid)-based antimicrobials (MBAs) that are effective against bacterial biofilms.

### BACKGROUND OF THE INVENTION

Antimicrobial resistance (AMR) is one of the main health concerns all over the globe. Antimicrobial treatment options are decreasing due to the increase of multidrug-resistant (MDR) organisms, which leads to critical clinical and industrial challenges (1, 2). According to predictions from the Centers for Disease Control and Prevention (CDC), by 2050, the mortality rate from AMR and MDR infections will be higher than all types of cancer combined (3). AMR has led to an urgent call for antimicrobial stewardship as well as a call for alternative anti-bacterial treatment(s) (4).

It is now understood that about 40-80% of bacterial cells on earth form or live in biofilms (5). The formation of biofilms is detrimental in several situations. For example, in food industries, pathogenic bacteria can form biofilms inside of processing facilities, leading to food spoilage, and endangering consumers' health (6). In hospital settings, biofilms have also been shown to persist on medical device surfaces and patient's tissues causing persistent infections (7). Furthermore, biofilms and persistent infections are linked to the high rate of mortality due to the increase of AMR. Thus, there is an urgent need for the prevention and control of harmful biofilms (7).

Hence, different antibiotics and anti-biofilm approaches are currently being pursued, some authors describing treatments using a single-agent antimicrobial that could be effective for both planktonic and biofilm forms of bacteria (8). However, such an approach is problematic since bacteria may quickly become tolerant or resistant to single antimicrobial agents (9). There is thus a need for combinations of antimicrobials that can work synergistically through diverse mechanisms, thereby making it more difficult for resistance to evolve. Furthermore, synergistic combinations may also allow for a lower antimicrobial dose of each individual compounds in the combination, and the combination thereby generating fewer side effects to patients (10).

Many metal(loid) s are known to have a very good antibacterial (16). For instance, silver has regained significant attention as an antibacterial and several patent documents define the use of silver as an antimicrobial (11). Examples of silver-containing products include silver-coated medical devices, implants, needles, bone implants, breast pumps, gloves, topical creams, deodorants, sanitary towel, contact lenses, etc. [see the review by Sim et al. (12)]. However, silver is now a victim of its popularity since the increasing number of silver-based products has led to increasing silver resistant microorganisms, which is a significant cause for serious concerns (13). Accordingly, there is also a need for anti-biofilm compositions that can increase the efficacy of silver while tackling the silver-resistance problem (14).

Recently, it was found that silver can demonstrate antibacterial synergism activities combined with other metal(loid)-based antimicrobials against Escherichia coli, Pseudomonas aeruginosa, and Staphylococcus aureus (15). However, that study was done with bacteria as planktonic form (not biofilm form). Since it is well established that bacterial biofilms are physiologically distinct from free-swimming planktonic cells (e.g., differences have been shown in motility, tolerance to the host immune system, polysaccharide production, antibiotic tolerance and global proteomic and transcriptomic profiles), that publication does not provide any evidence that the disclosed metal(loid)-based combinations would be effective against biofilms. Moreover, that publication does not provide any information about metalloid synergism efficacy against biofilms or if they can be effective against antibiotic resistance clinical isolates or not.

Accordingly, there is still an important need for effective anti-biofilm compositions, especially for antibiotic resistance isolates.

There is particularly a need for metal(loid)-based anti-biofilm compositions that combine a plurality of metal(loid) which together act synergistically against bacterial biofilms. Yet, there is a need for anti-biofilm compositions that will not only kill bacteria in a biofilm, but also prevent biofilms from initiating and proliferating.

There is also a need for both targeted anti-biofilm compositions that are effective against a specific bacteria species or groups, such as just Gram-negative or just Gram-positive bacteria. Also needed are broad-spectrum anti-biofilm compositions that are effective against all types of bacteria and/or a mixture of them.

There is further a need for anti-biofilm compositions that can be used for coating applications and that can confer anti-biofilm activity to a coated surface.

The present invention addresses these needs and other needs as it will be apparent from the review of the disclosure and description of the features of the invention hereinafter.

### BRIEF SUMMARY OF THE INVENTION

According to one aspect, the invention relates to an anti-biofilm composition comprising a combination of metal(loid) selected from the group consisting of: i) at least two metals; and ii) at least one metal and at least one metalloid; wherein said anti-biofilm composition is effective against bacterial biofilm.

According to another aspect, the invention relates to anti-biofilm composition comprising at least one of the following combinations of metal(loid): (i) silver nitrate and potassium tellurite; (ii) silver nitrate and hydrogen tetrachloroaurate (III); (iii) silver nitrate and nickel sulfate; (iv) silver nitrate and copper (II) sulfate; (v) silver nitrate and aluminium sulfate; (vi) silver nitrate and gallium nitrate; (vii) silver nitrate and sodium selenite; (viii) silver nitrate and gallium nitrate; and (ix) silver nitrate and zinc sulfate.

According to another aspect, the invention relates to an anti-biofilm composition comprising a combination of silver nitrate and potassium tellurite. In embodiments, that particular composition is especially effective against bacterial biofilms resulting from the growth of Gram-negative bacteria.

According to another aspect, the invention relates to an anti-biofilm composition comprising a combination of silver nitrate and zinc sulfate. In embodiments, that particular composition is especially effective against bacterial biofilms resulting from the growth of Gram-positive bacteria.

According to another aspect, the invention relates to a broad-spectrum anti-biofilm composition comprising a combination of silver nitrate, potassium tellurite and zinc sulfate.

According to another aspect, the invention relates to a method of disruption of biofilm on a surface, of removal of biofilm from a surface and/or of prevention of biofilm formation from a surface, comprising contacting said surface with an anti-biofilm composition as defined herein.

According to another aspect, the invention relates to method of disruption of biofilm present on a surface, and/or of removal of biofilm from a surface, comprising contacting the biofilm with a combination of metal(loid) selected from the group consisting of: i) at least two metal; and ii) at least one metal and at least one metalloid; wherein said combination is effective against bacterial biofilm.

According to another aspect, the invention relates to a method for conferring anti-biofilm activity to a surface, comprising contacting said surface with an anti-biofilm composition as defined herein.

According to another aspect, the invention relates to the use of an anti-biofilm composition as defined herein for conferring anti-biofilm activity to an article of manufacture and/or for providing anti-biofilm activity to a surface.

According to another aspect, the invention relates to a kit for disruption of biofilm on a surface, and/or of removal of biofilm from a surface, the kit comprising: (i) an anti-biofilm composition as defined herein; and (ii) at least one additional component selected from the group consisting of a user manual or instructions, a spray bottle, a mixing bottle, pen(s), marking sheets, boxes, holders, wipes, and cleaning solutions.

Additional aspects, advantages and features of the present invention will become more apparent upon reading of the following non-restrictive description of preferred embodiments which are exemplary and should not be interpreted as limiting the scope of the invention.

### BRIEF DESCRIPTION OF THE FIGURES

For the invention to be readily understood, embodiments of the invention are illustrated by way of examples in the accompanying figures.

FIGS. 1A and 1B are panels depicting analysis of synergism analysis against P. aeruginosa biofilm, in accordance with Example 1. FIG. 1A: Estimated fractional biofilm inhibitory concentration (FBIC) value for synergism biofilm inhibition efficacy. FIG. 1B: Estimated fractional biofilm eradication concentration (FBEC) values for synergism killing of cells in a biofilm efficacy. The synergistic efficacy of silver nitrate (Ag) with 8 other Metal(loid)-Based antimicrobials in three different media are defined. Detailed data and the most effective synergism components and concentrations are provided in Table 2. Data are presented as means of 2-5 (biological repeats) separate experiments over different days. Silver nitrate (AgNO₃; abbreviated to Ag], copper sulfate (CuSO₄; Cu), gallium nitrate ((Ga(NO₃)₃; Ga), nickel sulfate (NiSO₄; Ni), tetrachloroaurate (AuCl₄; Au), aluminium sulfate (Al₂(SO₄)₃; Al), sodium selenite (Na₂SeO₃; Se), potassium tellurite (K₂TeO₃; Te), and zinc sulfate (ZnSO₄; Zn).

FIGS. 2A and 2B are panels depicting the efficacy of an example mixed metal(loid)-based antibiotics (MBAs) formulations against clinical isolates, in accordance with Example 2. FIG. 2A: Minimum biofilm inhibition concentration (MBIC) of the example composition of Silver (Ag), Tellurite (Te), Ag—Te combination, Ciprofloxacin (Cip), and Gentamicin (Gen) on clinical isolates and indicator strains of P. aeruginosa (n=3). FIG. 2B: Synergism checkboard of synergism biofilm inhibitory of Ag—Te combination on clinical isolates and indicator strains of P. aeruginosa (n=3).

FIGS. 3A, 3B and 3C are panels depicting a synergism checkboard of synergism biofilm inhibitory, minimum biofilm inhibition concentration (MBIC) of example formulation of silver nitrate (Ag), zinc sulfate (Zn), Ag—Zn combination, on indicator strains of Staphylococcus aureus (n=3) in three different media Simulated Wound Fluid (SWF) (FIG. 3A), Muller Hinton Broth (MHB) (FIG. 3B), and Luria-Bertani (LB) (FIG. 3C), in accordance with Example 3.

FIGS. 4A to 4C are 3D bar graphs showing example recovery potency of P. aeruginosa after treatment with (Ag—Te) combination, recovery potency after 2 h (FIG. 4A), after 4 h (FIG. 4B), and after 24 h (FIG. 4C), in accordance with Example 4.

FIGS. 5A to 5C are 3D bar graphs showing example recovery potency of S. aureus after treatment with (Ag—Zn) combination, S. aureus recovery potency after 2 h (FIG. 5A), after 4 h (FIG. 5B), and after 24 h (FIG. 5C), in accordance with Example 5.

FIGS. 6A and 6B depicts the in vitro resistance acquisition survey for estimating the future chance of evolving resistance against Ag and/or Te in accordance with Example 5. Serial passage of growth as a screen for resistant P. aeruginosa PAO1 was carried out for 7 days in SWF supplemented with sublethal (1/8 dilution from the MIC) concentrations of Ag, Te, and Ag—Te mixture. FIG. 6A: Line graphs showing OD600 of serially passaged P. aeruginosa PAO1 in 30% SWF supplemented with 0.15 mM Ag and 0.015 mM Te, 0.015 mM Te, or 0.15 mM Ag. FIG. 6B: Panel showing pictures of a photo series of accumulating resistance in silver, tellurite, and combination. Strains of P. aeruginosa PAO1 were grown in sublethal (1/8th MIC) concentrations of Ag, Te or a mixture of the two antimicrobials, for 7 days. 10 uL aliquots of broth culture diluted 100-fold in saline were transferred to 1.2 mM Ag, 0.12 mM Te or 1.2 mM Ag/0.12 mM Te supplemented MHA to test each cultures resistance enrichment potency.

Further details of the invention and its advantages will be apparent from the detailed description included below.

### DETAILED DESCRIPTION OF EMBODIMENTS

In the following description of the embodiments, references to the accompanying figures are illustrations of an example by which the invention may be practised. It will be understood that other embodiments may be made without departing from the scope of the invention disclosed. Unless defined otherwise, all technical and scientific terms used herein have the same meaning as commonly understood by one of ordinary skill in the art to which the invention belongs.

### Anti-Biofilms Compositions

According to one aspect, the invention relates to an anti-biofilm composition comprising a combination of a) at least two metals; or b) at least one metal and at least one metalloid, the anti-biofilm composition being effective against bacterial biofilm.

As used herein the term “metal” refers to any element that readily forms ions in aqueous solution and has metallic bonds. Examples include, but are not limited to, silver, copper, gold, zinc, nickel gallium, aluminium, cobalt, bismuth, thallium, titanium, manganese, zirconium, iron, cadmium, tin, lead, mercury, vanadium, chromium and tungsten. The term “metal” encompasses metals ions such as Ag²⁺, Zn²⁺, Cu²⁺, Ni²⁺, Co²⁺, Al³⁺, Fe³⁺, etc.

As used herein the term “metalloid” refers to any element that exhibits some properties of metals and some properties of non-metals. Examples include, but are not limited to, tellurium, selenium, boron, silicon, germanium, arsenic, antimony, polonium, and astatine. The term encompasses metalloid ions typically in oxy ligands such as Te(IV) as TeO₃²⁻ or HTeO₃⁻, Te(VI) as TeO₄²⁻, Se (IV) as SeO₃²⁻, etc.

As used herein the term “metal(loid)” refers to a metal, a metalloid or both, indistinctly.

As used herein the term “biofilm” or “microbial biofilm” refers to any consortium of microorganisms as single or mixed species in which bacterial cells stick to each other or to a surface. Typicality these adherent cells are embedded within a “matrix” of biological molecules and polymers that can include but not limited to extra cellular polysaccharides (EPS), DNA, proteins, lipids, metabolites. The matrix limits the exposure of the bacteria to the host's immune system and/or antimicrobials. According to the present invention, biofilms may be composed of Gram-positive bacteria, Gram-negative bacteria, archaea, yeasts, fungi and/or a mixture of all types of microbes.

As used herein the term “anti-biofilm” or “effective against microbial biofilm” or “against biofilm” refers to any activity that can be against a biofilm, either killing the cells in a biofilm (eradication) or prevention of formation of the biofilm. According to the present invention, an anti-biofilm agent or composition may have one or more of the following activities: prevention of initiation of biofilm formation; prevention of microbial cell attachment on a surface; prevention of bacterial cell division and proliferation if the microbial cell becomes attached; killing of cells and eradication of a preformed or pre-existing microbial biofilm; killing microbial cells forming a biofilm; preventing recovery of microbe(s) from a biofilm; preventing recovery of microbial cells is a fresh media after prior contacting with the anti-biofilm composition; preventing reformation of a biofilm by microbial cells previously removed from the biofilm, etc. Similarly, as used herein the “eradication of biofilm” and “prevention of biofilm formation” broadly encompasses one or more relevant anti-biofilm related activities among those listed hereinabove. In embodiments the anti-biofilm composition is “microbicidal” or “bactericidal”, i.e. it prevents propagation and/or recovery of the microbe(s) (e.g. bacteria) such that the microbe cannot recover after being contacted with the anti-biofilm composition for a certain period of time.

In embodiments, the combination of metal(loid) is selected such that the metal and/or metalloid act synergistically against bacterial biofilms. As used herein the term “act synergistically” or “synergistic activity”, when used in connection with the terms “metal” or “metalloid”, refers to the interaction or cooperation of two or more metal and/or metalloid to produce a combined anti-biofilm activity that is greater than the sum of their separate effects. As described herein, having a synergistic activity against biofilms and/or microbes may allow for a lower antibacterial dose (e.g., reduced dose) of each individual metal(loid) in the combination, the combination thereby generating less side effects and/or less toxicity (e.g., side effects might be caused by high doses of metal(loid), but in a synergism manner, a lower dosage has same anti-biofilm efficacy but with lower side effects to the host). Having a synergistic activity against biofilms and/or bacteria may also reduce the likelihood of developing antibiotic resistance (e.g., reducing resistance against a given metal(loid) by using the combination of two or more metal(loid), when compared to a composition comprising only one of the two metal(loid)). Accordingly, in embodiments, the term “act synergistically” or “synergistic activity” encompasses an anti-biofilm activity that is at least 2×, 3×, 4×, 5×, 6×, 7×, 8×, 9×, 10×, 11×, 12×, 13×, 14×, 15×, 16×, 17×, 18×, 19×, 20×, 21×, 22×, 23×, 24×, 25×, 30×, 35×, 40×, 45×, 50×, 75×, 100× or more, greater than antibiofilm activity of a individual metal(loid).

By way of example, as demonstrated hereinafter in the Examples section, it was found that an Ag—Te combination treatment decreased the effective concentration of both agents, drastically. For biofilm inhibition with Ag, minimum biofilm inhibition concentration (MBIC) in simulated wound fluid (SWF) was 0.25 mM, whereas, when used in combination with Te, the effective concentration of Ag decreased to 0.015 mM (i.e., about 17× less). The MBIC for Te was 0.5 mM, while Ag—Te combination decreased the effective concentration of Te to 0.063 mM (about 8× less). There was a similar outcome for biofilm eradication. Minimum biofilm eradication concentration (MBEC) for both Ag and Te was 0.5 mM, while Ag—Te combination decreased the effective concentration of both agents to 0.065 mM (about 8× less).

Preferably, the anti-biofilm composition comprises at least one, preferably two, three or more of the following activities:
prevention of microbial cell attachment on a surface;
prevention of initiation of biofilm formation;
eradication of a preformed or pre-existing microbial biofilm;
killing microbial cells forming the biofilm;
preventing recovery of microbial cells in a fresh media after exposure with the anti-biofilm composition;
preventing propagation and/or recovery of microbial cells removed from the biofilm;
preventing reformation of a biofilm by bacterial cells previously removed from the biofilm;
effective against clinical antibiotic-resistant strains of microbes;
elimination of persister cells known to be extremely antimicrobial-resistant;
significant decrease in the effective dose which reduces the side effects to the host; and
drastic elimination on the likelihood of getting resistance in the future.

In embodiments, the anti-biofilm composition comprises a combination of at least two metal(loid) as defined in Table 1:

TABLE 1
Combinations of metal(loid) in the anti-biofilm composition

| | | Metal | | | | | | | | | | | Metalloid | |
|---|---|---|---|---|---|---|---|---|---|---|---|---|---|---|
| | | Ag | Zn | Ga | Cu | Au | Ni | Al | Co | Ti | Bi | V | Se | Te |
| Metal | Ag | — | X | X | X | X | X | X | X | X | X | X | X | X |
| | Zn | | — | X | X | X | X | X | X | X | X | X | X | X |
| | Ga | | | — | X | X | X | X | X | X | X | X | X | X |
| | Cu | | | | — | X | X | X | X | X | X | X | X | X |
| | Au | | | | | — | X | X | X | X | X | X | X | X |
| | Ni | | | | | | — | X | X | X | X | X | X | X |
| | Al | | | | | | | — | X | X | X | X | X | X |
| | Co | | | | | | | | — | X | X | X | X | X |
| | Ti | | | | | | | | | — | X | X | X | X |
| | Bi | | | | | | | | | | — | X | X | X |
| | V | | | | | | | | | | | — | X | X |
| Metalloid | Se | | | | | | | | | | | | — | — |
| | Te | | | | | | | | | | | | | — |

The anti-biofilm composition according to the present invention may be formulated as powder form, a water-soluble composition, as a slurry or as a suspension.

In embodiments, the metal or metalloid is present in the composition as a salt. The salt may comprise suitable counter ion(s), for instance, a counter ion selected from K⁺, Na⁺, NO₃²⁻, SO₄²⁻, PO₄²⁻, Cl⁻, and Br⁻. Particular examples of metal(loid) salts that May be useful in accordance to the present invention include, but are not limited to, silver nitrate (AgNO₃), silver phosphate (Ag₃PO₄), silver nitrite (AgNO₂), silver lactate (CH₃CH(OH)COOAg), silver sulfate (Ag₂SO₄), silver iodide (AgI), silver fluoride (AgF), silver bromide (AgBr), zinc sulfate (ZnSO₄), zinc fluoride (ZnF₂), zinc iodide (ZnI₂), zinc bromide (ZnBr₂), zinc nitrate (Zn(NO₃)₂), zinc acetate (Zn₄O(CH₃CO₂)₆), nickel sulfate (NiSO₄); copper (II) sulfate (CuSO₄), copper (II) chloride (CuCl₂), copper (ii) fluoride (CuF₂), copper (II) iodide (CuI₂), copper (II) bromide (CuBr2), copper (II) acetate (Cu(CH₃COO)₂), aluminium sulfate (Al₂(SO₄)₃), gallium nitrate (Ga(NO₃)₃, gallium iodide (GaI₃), gallium bromide (GaBr₃), gallium chloride (GaCl₃), gallium triacetate (Ga(C₂H₃O₂)₃). hydrogen tetrachloroaurate (III) (HAuCl₄), potassium tellurite (K₂TeO₃), sodium selenite (Na₂SeO₃), and mixtures thereof.

In embodiments, the anti-biofilm composition comprises at least one metal(loid). In embodiments, the anti-biofilm composition comprises at least one of silver nitrate, zinc sulfate, and potassium tellurite. In embodiments, the anti-biofilm composition comprises at least silver nitrate in combination with one of the other metal(loid) s. In embodiments, the anti-biofilm composition comprises at least two of silver nitrate, zinc sulfate, and potassium tellurite (e.g., silver nitrate+zinc sulfate, zinc sulfate+potassium tellurite, silver nitrate+potassium tellurite, and silver nitrate+zinc sulfate+potassium tellurite). In preferred embodiments, the anti-biofilm composition comprises at least silver nitrate in combination with one of the other metal(loid) s.

In embodiments, the anti-biofilm composition comprises at least one of the following combinations:
(i) silver nitrate and potassium tellurite;
(ii) silver nitrate and hydrogen tetrachloroaurate (III);
(iii) silver nitrate and nickel sulfate;
(iv) silver nitrate and copper (II) sulfate;
(v) silver nitrate and aluminium sulfate;
(vi) silver nitrate and gallium nitrate;
(vii) silver nitrate and sodium selenite;
(viii) silver nitrate and gallium nitrate; and
(ix) silver nitrate and zinc sulfate.

In embodiments, the concentration or amount of each of the metal(loid) in the anti-biofilm composition is selected to obtain a desired activity, preferably a synergistic activity. In embodiments the anti-biofilm composition comprises at least one of the following combinations:
(i) about 1% w/w metal to about 99% w/w metal, or about 10% w/w metal to about 90% w/w metal;
(ii) about 1% w/w metal to about 99% w/w metalloid, about 10% w/w metal to about 90% w/w metalloid.

In embodiments, the composition comprises:
0.015 mM Ag+0.063 mM Te;
0.065 mM Ag+0.063 mM Te;
0.5 mM Ag+0.5 mM Te;
0.065 mM Ag+0.065 mM Te;
0.031 mM Ag+0.25 mM Zn;
0.125 mM Ag+1 mM Zn;
0.007 mM Ag+3 mM Te;
0.065 mM Ag+0.125 mM Au;
0.031 mM Ag+2 mM Cu;
0.015 mM Ag+0.125 mM Ni;
0.015 mM Ag+0.1 mM Al; or
0.065 mM Ag+25 mM Se.

In embodiments, the amount of metal(loid) in the anti-biofilm composition is in accordance with the concentrations provided in the Table 2 and 3 for Gram-negative (e.g., P. aeruginosa) and in Table 4 for example Gram-positive (e.g., S. aureus).

In embodiments, the amount of metal(loid) in the anti-biofilm composition for different media conditions (MHB, LB, SWF) is in accordance any one of Tables 2, 3 and 4. The three different media provide the bacteria different physiological states that are found in a variety of real-life situations providing low nutrient to the very nutrient-rich environment.

In embodiments the anti-biofilm composition is effective against a bacterial biofilm comprising Gram-positive bacteria, Gram-negative bacteria and/or a mixture of both types of bacteria.

In embodiments, the anti-biofilm composition is effective against Gram-negative bacteria (e.g., against a bacterial biofilm comprising gram-negative bacteria), preferably at least against P. aeruginosa. In embodiments, the anti-biofilm composition is also effective against other Gram-negative bacteria including, but not limited to, Acinetobacter baumannii, Escherichia coli, Klebsiella pneumoniae, Proteus mirabilis.

In embodiments, the anti-biofilm composition is effective against Gram-positive bacteria (e.g., against a bacterial biofilm comprising gram-positive bacteria) preferably at least against Staphylococcus aureus. In embodiments, the anti-biofilm composition is also effective against other gram-positive bacteria including, but not limited to, Enterococcus faecalis, Enterococcus Vancomycin-Resistant faecium, Enterococcus (VRE), Staphylococcus epidermidis, Streptococcus pneumoniae and, Streptococcus pyogenes.

In embodiments, the anti-biofilm composition is directed against Gram-positive bacteria, including bacterial biofilms comprising and/or resulting from the growth of a Gram-positive bacteria, and the anti-biofilm composition comprises at least silver nitrate and zinc sulfate.

In embodiments, the anti-biofilm composition is directed against Gram-negative bacteria, including bacterial biofilms comprising and/or resulting from the growth of a gram-negative bacteria, and the anti-biofilm composition comprises at least silver nitrate and potassium tellurite.

In embodiments, the anti-biofilm composition is directed against both Gram-positive and Gram-negative bacteria, particularly against bacterial biofilms comprising and/or resulting from the growth of both Gram-positive and Gram-negative bacteria, and the anti-biofilm composition comprises at least silver nitrate, zinc sulfate and potassium tellurite.

In embodiments, the anti-biofilm composition is effective against antimicrobial-resistant bacteria that are resistant to one or more antimicrobial(s) (e.g., silver (e.g., silver nitrate), tellurite, methicillin, vancomycin, ciprofloxacin, gentamicin, etc.). In embodiments, the anti-biofilm composition is effective against clinical isolates, including clinical isolates that are resistant to one or more antimicrobials. In one embodiment the anti-biofilm composition is effective against one or more of the following indicator strains: P. aeruginosa ATCC 27853, PAO1, and Staphylococcus aureus ATCC 25923.

The compositions of the present invention may further include additional anti-biofilm active ingredients (e.g., antibiotics, chemicals, antioxidant, anti-inflammation, anti-corrosion, etc.) and/or non-active additives, such as fragrance, colors, inorganic salts, inorganic acids, sequestrants, organic solvents, fillers, rheology modifiers, thickener, etc.

The compositions according to the present invention may be formulated in accordance with desired uses, including but not limited to coating applications, as disinfectants or cleaner applications (e.g., spray, wetted wipes, dipping solution, etc.), impregnated into textiles (e.g., wound dressings, personal protective clothing) as preservatives of cosmetic formulations (e.g., lotions, topical creams and deodorants), and the like.

The compositions according to the present invention may be formulated in accordance with desired field(s) of application. For instance, requirements for human-related uses are different from industrial-related uses since both fields of endeavour are subjected to different requirements and safety regulations [e.g., in the United States medical devices, drugs and food products for humans and veterinary are governed by the Food and Drug Administration (FDA)]. Therefore metal(loid) and/or related compounds (e.g., salts) may thus be selected in accordance with the desired field of uses and metal(loid) that are acceptable for medical industrial applications (e.g., oil pipes, ship hauls, vehicles, etc.) may not be acceptable for human and/or veterinary applications. Without wishing to be bound by any example, arsenic or cadmium for instance may be totally acceptable for uses in the oil industry (e.g., coating of pipes) but it would be totally prohibited for medical applications because of their well-known toxicity to humans and animals. Metal(loid) (s) that may not be suitable for medical purposes but possibly suitable to some industries include, but are not limited to, cadmium (Cd), lead (Pb), mercury (Hg), tin (Sn), arsenic (As) and tungsten (W). It is within the skill of those in the art to be informed about the safety regulations and to select accordingly proper combinations of metal(loid) s that are safe and acceptable for a given industry.

### Methods and Uses

The metal(loid) compositions according to the present invention have very good antimicrobial activity, and therefore, they have excellent potential to address different clinical and industrial needs.

According to additional aspects, the invention relates to the uses of the compositions according to the present invention, particularly for acting against bacterial biofilms. Additional aspect concerns the uses of anti-biofilm compositions as defined herein for conferring anti-biofilm activity to an article of manufacture and/or for providing anti-biofilm activity to a surface. For instance, metal(loid)-based antibiotics can be an appropriate option for high touch surface coating because of their long half-life.

According to particular aspects, the invention relates to methods of eradication of biofilm on a surface, to methods of disruption of biofilm from a surface, to methods of removal of biofilm from a surface and/or to prevention of biofilm formation from a surface, these methods comprising contacting the surface with an anti-biofilm composition as defined herein.

In one embodiment, the invention comprises a method of disruption of biofilm present on a surface, and/or of removal of biofilm from a surface, comprising contacting the biofilm with a combination of metal(loid) selected from:
a) at least two metals; and
b) at least one metal and at least one metalloid;
   wherein the combination is effective against bacterial biofilm.

As described hereinbefore, the combination is preferably selected such that the metal and/or metalloid acts synergistically against bacterial biofilms. Additional features of the combination are as defined hereinabove for the anti-biofilm compositions.

In embodiments the surface is made up of a material selected from the group consisting of plastic, polymer, stainless steel, steel, metal, ceramic, biopolymer, concrete, leather, fabric, wood and synthetic wood.

In embodiments the surface is a surface of a furniture, partition curtains, handrails, doors, doorknobs, walls, floors, elevators, air vents, air filters, pipes, vessels, medical devices, kitchen items, implants, needles, bone implants, breast pumps, medical gloves, personal protective equipment, wound dressings, surgical equipment, clothing, electronic devices, cosmetic lotions (topical creams and deodorants), sanitary towels, and/or contact lenses.

The present invention may find uses in various environments and the surface (contaminated with a biofilm or at risks of bacterial contamination) may technically be anywhere, for instance in a hospital, a care setting, a restaurant, a food-processing facility, a slaughterhouse, a laboratory, a vehicle, a boat, a manufacture, an industrial cooling system (e.g., a cooling tower), a petroleum product storage (e.g., thank or reservoir), a petroleum product distribution system (e.g. pipe), etc.

In embodiments, the contacting is for at least 5 minutes, or at least 10 minutes, or at least 15 minutes, or at least 20 minutes, or at least 25 minutes, or at least 30 minutes, or at least 1 hour, or at least 5 hours, or at least 12 hours or at least 24 hours or more.

According to another aspect, the invention relates to a method for conferring anti-biofilm activity to a surface. In one embodiment the method comprises contacting the surface with an anti-biofilm composition as defined herein.

In embodiments, the method comprises spraying or brushing the surface with the anti-biofilm composition. In embodiments, the method further comprises letting the surface dry for about 2 minutes to about 1 hour. Time may depends on various factors, for instance the type of surface, the compounds in the composition and/or the nature of carrying polymer/solvent used for the coating.

Many types of surfaces may benefit of having an anti-biofilm coating. For instance, the surface may be found in a hospital, a care setting, a restaurant, a food-processing facility, a slaughterhouse, a laboratory, on a vehicle, on a boat, water and petroleum pipes, etc. The surface may be the surface of a furniture, partition curtains, handrails, doors, doorknobs, walls, floors, elevators, air vents, air filters, pipes, vessels, a vehicle body or chassis, a ship haul, medical devices, implants, needles, bone implants, breast pumps, medical gloves, mask, clothes, sanitary towels, and/or contact lenses.

In embodiments, the coating preferably confers long-lasting anti-biofilm activity against Gram-positive bacteria, against Gram-negative bacteria and/or against both types of bacteria.

### Kits

A further aspect of the invention relates to kits. The kits of the invention may be useful for the practice of the methods of the invention, particularly for acting against biofilms, for the disruption of biofilm on a surface, for eradication of biofilm from a surface, for cleaning a surface/skin/surgery site, food processing, for oral mouthwash, and disinfectant, fouled pipes, etc.

A kit of the invention may comprise one or more of the following components: (i) a composition as defined herein; and (ii) at least one additional component, including but not limited to: a user manual or instructions, a spray bottle, a mixing bottle, a mixing pump, pen(s), marking sheets, boxes, holders, wipes, and cleaning solutions, etc.

In the kit, the composition of the invention may be formulated as a liquid concentrate to be diluted before use.

Kits in accordance with the present invention may comprise two different bottles (e.g., separate bottles for different metal(loid)), each bottle having a different concentrated solution to be mixed and diluted with water in order to provide a final ready-to-use solution. Such kits may further comprise, among other things instructions for mixing the concentrated solutions in order to obtain the ready-to-use solution, a mixing vessel, a protective mask, protective gloves, etc.

Those skilled in the art will recognize or be able to ascertain, using no more than routine experimentation, numerous equivalents to the specific procedures, embodiments, claims, and examples described herein. Such equivalents are considered to be within the scope of this invention and covered by the claims appended hereto. The invention is further illustrated by the following examples, which should not be construed as further or specifically limiting.

### EXAMPLES

The present examples provide examples of metal(loid) based antimicrobials (MBAs) combination approach concentrations to combat bacterial biofilms. Studies were carried out using Pseudomonas aeruginosa for the Gram-negative examples and Staphylococcus aureus for the Gram-positive examples. These bacteria were selected because they are the most prolific biofilm-forming bacteria in model systems and they both contribute to many diseases in the biofilm state (17-19). Effectiveness against these harmful biofilms, therefore, support the robustness of the present invention.

### Experimental Details

### Bacterial Strains, Growth Conditions and Chemicals

Three indicator strains including P. aeruginosa ATCC 27853, S. aureus ATCC 25923, P. aeruginosa PAO1, as well as 39 clinical isolates were used. Out of 39 clinical isolates (20 were isolated from cystic fibrosis and 19 burn wounds), 17 (43%) of them were resistant to the most common antibiotics (ciprofloxacin or gentamicin). Out of these 39, 15 (38%) isolates were resistant to gentamicin while 2 (5%) isolates were resistant to ciprofloxacin. None of the isolates was resistant to both antibiotics.

Bacterial strains were stored at −70° C. in Micro-bank™ vials as described by the manufacturer (ProLab Diagnostics, Richmond Hill, ON, Canada). Three different media were used in this study: Luria-Bertani Broth (LB, VWR chemicals, Lot #190756384) Mueller-Hinton Broth (MHB, BD Bacto, Oxoid, Basingstoke, UK Cat #X296B), and simulated wound fluid (SWF) [50% peptone water (0.85% NaCl, 0.5 g peptone per 500.0 mL): 50% foetal calf serum (GIBCO, Thermo Fisher Scientific, Waltham, MA, USA, Lot #2212202RP)] were used as the growth medium and for susceptibility testing media in this example (20, 21).

Nine metal(loid)-based antibiotics (MBAs) including, silver nitrate (AgNO₃, Sigma-Aldrich, St Louis, MO, USA Lot #39F-3539), copper sulfate (CuSO₄, Sigma-Aldrich, St Louis, MO, USA, Cat #C1297-100G), gallium nitrate (Ga(NO₃)₃·H₂O Sigma-Aldrich, St Louis, MO, USA, Cat #289892-25G) and nickel sulfate (NiSO₄·6H₂O, Sigma-Aldrich, St Louis, MO, USA Lot #68H0027), hydrogen tetrachloroAurate trihydrate (AuCl₄·3H₂O, Alfa Aesar Co, Canada, Lot #U17G046), aluminium sulfate (Al₂(SO₄)₃·H₂O) (Norwood, OH, USA Cat #UCC000TA8), sodium selenite (Na₂SeO₃, Alfa Aesar, Ward Hill, MA, USA, Lot #61400984), potassium tellurite (K₂TeO₃, Sigma-Aldrich, St Louis, MO, USA, Cat #P0677-25G), and zinc sulfate (ZnSO₄·7H₂O) were received from Fisher Scientific (Fair Lawn, NJ, USA, Lot #723689). Stock solutions for Ag were made up to 50 mM and for all other MBA was made up to 1 M; working solutions for Ag were made up to 5 mM and for all other MBA was made up to 100 mM in distilled and deionized (dd) H₂O. All stock metal(loid) dilutions were stored in glass vials stored at room temperature and dark place for no longer than 2 weeks. No more than 30 min before experimental use, working solutions were made from stock metal(loid) solutions in equal amounts of each media. Antimicrobial assays were performed in a 96-well plate (the challenge plate), serial dilutions of each metal(loid), with a dilution factor of 2, were prepared; reservation of the first column served as a negative control (Media, 0 mM metal salt and no bacteria) and the last column served as a positive control (Media and bacteria, 0 mM metal salt and with bacteria).

For the MIC, stored bacteria (in −70° C.) were sub-cultured two times overnight (O/N) at 37° C. on agar plates to obtain pure single colony. Desired concentrations of metal salt stock were added to 96 wells (75 μL), then 75 μL of 1.0×10⁶ CFU/ml bacteria were added to each well and the plate was incubated in a shaker incubator for 24 hours at 37° C. (21). The last well which had no bacterial growth was defined as the MIC.

For the MBC and recovery potency of bacteria. At the end of the MIC determination experiment, 10 μL of each MIC well was transferred in 140 μL of the same fresh media in a new 96 plate and incubated in a shaker incubator for 24 hours at 37° C. The last well that had no bacterial growth was defined as the MBC. For analyzing of recovery potency of exposed bacteria with metals, plates were read at 2, 4, and 24 hours.

### Synergism High-Throughput Susceptibility Testing

This study was formatted as a checkerboard (96-well microtiter plates) including one column only with media without bacteria and MBAs (as a negative control), and one column without MBA, with media, and bacteria (as a growth and positive controls control), 10 different concentrations of Te alone, 8 different concentrations of Ag alone, and combination of Te and Ag at 80 different concentrations (22, 23). For each checkboard analysis, the same MIC and MBC steps indicated above were conducted for surveying bacteriostatic, bactericidal, and reading synergism effects of MBAs combinations.

The synergistic interactions rules suggested by the American Society for Microbiology for the testing of planktonic cells are used here for the MIC, MBC, MBIC and MBEC synergism data obtained (22). The fractional inhibitory concentration (FIC), fractional bactericidal concentration (FBC), fractional biofilm inhibitory concentration (FBIC) and fractional biofilm eradication concentration (FBEC) index for each combination of antimicrobial agents was calculated with the following formula:

FIC=MIC antibiotic A in combination/MIC antibiotic A alone+MIC antibiotic B in combination/MIC antibiotic B alone.

FBC=MBC antibiotic A in combination/MBC antibiotic A alone+MBC antibiotic B in combination/MBC antibiotic B alone.

FBIC=MBIC antibiotic A in combination/MBIC antibiotic A alone+MBIC antibiotic B in combination/MBIC antibiotic B alone.

FBEC=MBEC antibiotic A in combination/MBEC antibiotic A alone+MBEC antibiotic B in combination/MBEC antibiotic B alone.

To evaluate antimicrobial interactions, the lowest FIC/FBC/FBIC/FBEC index method was used, as described by Bonapace et al. (24). Finally, FIC/FBC/FBIC/FBEC were interpreted as following: FIC/FBC/FBIC/FBEC <0.8=Synergy, FIC/FBC/FBIC/FBEC ≥0.8 and ≤1.2=Partial-synergy, FIC/FBC/FBIC/FBEC >1.2=Antagonistic.

### Example Data

Prevention of biofilm formation by microtiter plate method. Briefly, −70° C. stored bacteria were sub-cultured at 37° C. overnight (O/N) to get a pure single colony, 75 L of desired concentration of metals (loid) (provided in the media) added to 96 wells, 75 μL of 1.0×106 CFU/ml bacteria added in each well and incubated 48 hours at 37° C. in a microplate shaker incubator at 150 rpm. The planktonic cells and the spent medium were discarded, and the adhered biomass was rinsed two times with distilled water. The biofilm biomass and minimum biofilm inhibitory concentration (MBIC) was determined by crystal violet assay (see below). The last well which had no bacterial biofilm and zero OD600 absorption considered MBIC. Results from at least three separate biological replicates were averaged.

Crystal violet assay. Crystal violet staining was used to quantify the amount of biomass in the biofilm prevention and biofilm eradication assays. Following MBAs treatment, the microplates (in the biofilm prevention method) or pegged lids from the CBD (in the biofilm eradication method) were rinsed twice in 200 μl of 0.9% saline. By using a procedure similar to the one described previously by O'Toole (25), the biofilms were stained with 200 μl of a 0.1% crystal violet solution for 30 min. Following staining, the microplates and pegs were washed with 200 μl ddH₂O three times to remove excess dye. Quantification of the biofilm was performed by sonication using a 250HT™ ultrasonic cleaner (VWR International), set at 60 Hz for 10 min into 200 μl of 70% ethanol and reading the absorbance at 600 nm using 70% ethanol as the blank.

Biofilm cultivation. Biofilms were grown in a Calgary biofilm device (CBD; commercially available as the MBEC physiology and genetics assay [Innovotech Inc., Edmonton, Alberta, Canada]), as originally described by Ceri et al. (26). Starting from cryogenic stocks, P. aeruginosa ATCC 27853 was streaked out twice on TSA. One hundred fifty microliters of 1.0×10⁷ CFU/ml bacteria inoculum were transferred into each well of a 96-well microtiter plate, and the sterile peg lid of the CBD was inserted into the plate. The inoculated device was then placed on a microplate shaker at 150 rpm for 24 h of incubation at 37° C. and 95% relative humidity.

Eradication of established biofilms. After developing a biofilm on CBD, the pegs were rinsed twice with 0.9% saline to wash away planktonic bacteria, then placed into a 96-well microtiter plate containing twofold serial dilutions of the MBAs on the 150 μl of each media; a column was reserved for bacterial growth in the absence of the metal(loid) salts. The microtiter plate was then incubated for 24 h in a humidified incubator at 37° C. on a gyrorotary shaker at 150 r.p.m. This treatment was used to determine the minimum biofilm eradication concentration (MBEC) of each MBAs (27). The last well which had no bacterial biofilm and OD absorption considered MBEC.

### Example 1: MBAs Combinations for the Prevention and Eradication of Pseudomonas aeruginosa Biofilms

Table 2 provides the results of the testing of the different MBAs tested against P. aeruginosa in various media. As can be seen, the most effective MBAs for P. aeruginosa biofilm inhibition and eradication are Ag and Te. Yet, many other combinations are also quite effective.

TABLE 2
Prevention and eradication of P. aeruginosa biofilm:
Metal-based antibiotics minimum biofilm inhibitory
concentration (MBIC) and minimum biofilm eradication concentration
(MBEC) against P. aeruginosa.

| | | Media | | |
|---|---|---|---|---|
| | MBAs | MHB | LB | SWF |
| Inhibition of | Ag | 0.03 | 0.125 | 0.25 |
| P. aeruginosa | | (0.03-0.125) | (0.065-0.25) | (0.125-1) |
| biofilm | Cu | 8 | 8 | 8 |
| (MBIC) | | (4-32) | (4-32) | (4-16) |
| (mM) | Ga | 12.5 | 12.5 | 25 |
| [in 48 h] | | (6.25-50) | (6.25-50) | (6.25-50) |
| | Zn | 8 | 8 | 32 |
| | | (4-8) | (4-16) | (4-64) |
| | Te | 0.5 | 0.5 | 0.5 |
| | | (0.125-1) | (0.25-1) | (0.125-1) |
| | Se | >50 | >50 | >50 |
| | Al | 8 | 8 | 32 |
| | | (4-16) | (4-16) | (4-64) |
| | Ni | 8 | 16 | 2 |
| | | (4-16) | (4-16) | (1-16) |
| | Au | 0.25 | 0.5 | 0.25 |
| | | (0.25-1) | (0.25-1) | (0.25-1) |
| Eradication of | Ag | 0.03 | 0.065 | 0.5 |
| P. aeruginosa | | (0.015-0.5) | (0.065-0.125) | (0.125-1) |
| biofilm | Cu | 6.25 | 6.25 | 6.25 |
| (MBEC) | | (6.25-32) | (3-16) | (6.25-32) |
| (mM) | Gal | 3 | 3 | 12.5 |
| [in 24 h] | | (1.5-6.25) | (1.5-6.25) | (3-25) |
| | Zn | 6.25 | 3 | 6.25 |
| | | (6.25-32) | (1.5-6.25) | (1.5-6.25) |
| | Te | 0.5 | 0.5 | 0.5 |
| | | (0.25-1) | (0.25-0.5) | (0.125-1) |
| | Se | >50 | >50 | >50 |
| | Al | 16 | 16 | 16 |
| | | (8-32) | (4-16) | (4-16) |
| | Ni | 6.25 | 6.25 | 6.25 |
| | | (6.25-32) | (6.25-12) | (3-122) |
| | Au | 0.25 | 0.25 | 1 |
| | | (0.25-1) | (0.125-1) | (0.125-2) |

Value ranges are given in parentheses
MHB = Mueller-Hinton broth, LB = Luria-Bertani, SWF = simulated wound fluid,
the lowest MBIC and MBEC are highlighted.

Among the 1,920 possible combinations of MBAs concentrations, the most effective anti-biofilm combinations were tested for synergy. As demonstrated in Table 3, many combinations showed synergism in prevention and eradication potency for P. aeruginosa biofilms.

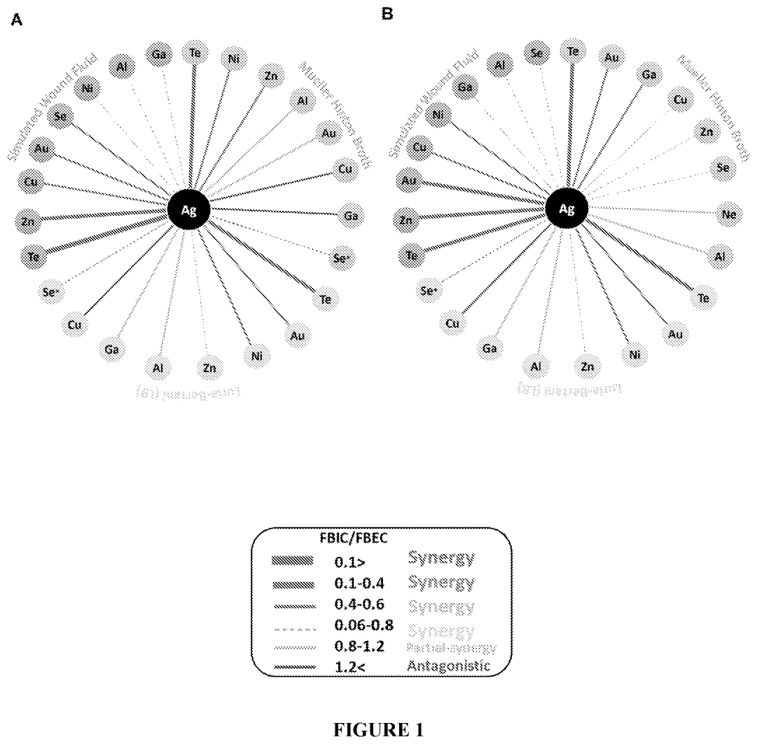

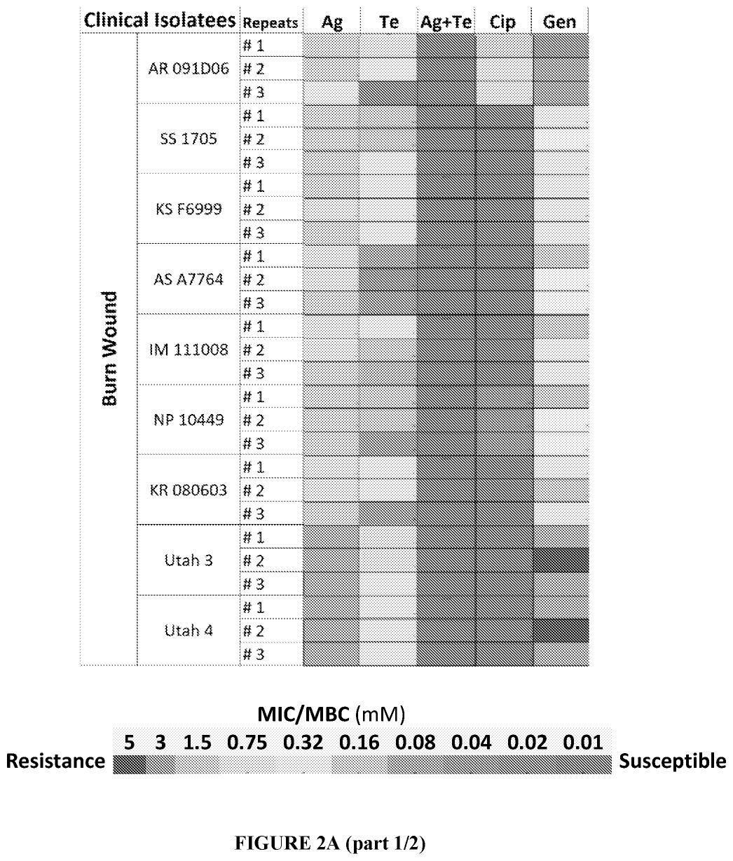

As illustrated in FIG. 1, the combination of Ag with Te was found to be the most effective anti-biofilm combination against P. aeruginosa with the lowest FBIC (0.093) (the highest level of synergism for biofilm inhibition) and the lowest FBEC (0.24) (the highest synergism for biofilm eradication) in SWF (0.015 mM Ag+0.063 mM TeI) or MHB (0.015 mM Ag+0.008 mM TeI), respectively.

TABLE 3
Synergism analysis against P. aeruginosa biofilm; estimated fractional biofilm inhibitory
concentration (FBIC) and estimated fractional biofilm eradication concentration (FBEC) values
for combinations of Metal(loid)-Based antimicrobials against P. aeruginosa biofilm.

| | Meta-based | | Biofilm inhibition synergism | | | Biofilm Eradication synergism | | |
|---|---|---|---|---|---|---|---|---|
| | antibiotic | | activity (FBIC) | | | activity (FBEC) | | |
| | agent | agent | | | Conc. | | | Conc. |
| Media | A | B | FIC | Interpretation | (mM) | FEC | Interpretation | (mM) |
| SWF | Ag | Te | 0.093 | Synergy | 0.015 Ag + | 0.27 | Synergy | 0.065 Ag + |
| | | | | | 0.063 Tel | | | 0.063 Tel |
| LB | Ag | Te | 0.36 | Synergy | 0.03 Ag + | 0.37 | Synergy | 0.015 Ag + |
| | | | | | 0.06 Tel | | | 0.125 Tel |
| MHB | Ag | Te | 0.37 | Synergy | 0.015 Ag + | 0.24 | Synergy | 0.015 Ag + |
| | | | | | 0.125 Tel | | | 0.008 Tel |
| SWF | Ag | Zn | 0.38 | Synergy | 0.065 Ag + | 0.36 | Synergy | 0.125 Ag + |
| | | | | | 4 Zn | | | 1.5 Zn |
| SWF | Ag | Cu | 0.5 | Synergy | 0.065 Ag + | 0.48 | Synergy | 0.007 Ag + |
| | | | | | 2 Cu | | | 3 Cu |
| LB | Ag | Au | 0.5 | Synergy | 0.03 Ag + | 0.48 | Synergy | 0.03 Ag + |
| | | | | | 0.125 Au | | | 0.065 Au |
| LB | Ag | Ni | 0.51 | Synergy | 0.065 Ag + 4 Ni | 0.7 | Partially- | 0.015 Ag + |
| | | | | | 0.032 Ag + 8 Ni | | Synergy | 3 Ni |
| MHB | Ag | Ni | 0.53 | Synergy | 0.031 Ag + 0.5 | 0.98 | Synergy | 0.25 Ag + |
| | | | | | Ni | | | 3 Ni |
| SWF | Ag | Au | 0.58 | Synergy | 0.065 Ag + | 0.37 | Synergy | 0.03 Ag + |
| | | | | | 0.016 Au | | | 0.125 Au, |
| | | | | | | | | 0.015 Ag + |
| | | | | | | | | 0.25 Au |
| MHB | Ag | Zn | 0.58 | Synergy | 0.065 Ag + | 0.72 | Synergy | 0.015 Ag + |
| | | | | | 0.5 Zn | | | 3 Zn |
| SWF | Ag | Se | <0.62 | Synergy | 0.125 Ag + | <0.77 | Synergy | 0.065 Ag + |
| | | | | | 12.5 Se | | | 25 Se |
| LB | Ag | Zn | 0.62 | Synergy | 0.015 Ag + | 0.96 | Partially- | 0.03 Ag + |
| | | | | | 4 Zn | | Synergy | 1.5 Zn |
| SWF | Ag | Ni | <0.75 | Synergy | 0.25 Ag + 1 Ni | 0.49 | Synergy | 0.007 Ag + |
| | | | | | | | | 3 Ni |
| SWF | Ag | Al | 0.75 | Synergy | 0.125 Ag + | 0.75 | Synergy | 0.25 Ag + 25 |
| | | | | | 12.5 Al | | | Al |
| SWF | Ag | Ga | 0.75 | Synergy | 0.25 Ag + | 0.75 | Synergy | 0.25 Ag + |
| | | | | | 12.5 Ga | | | 6.25 Ga |
| LB | Ag | Al | 0.9 | Partially- | 0.25 Ag + 25 Al | 1 | Partially- | 0.25 Ag + 50 |
| | | | | Synergy | | | Synergy | Al |
| LB | Ag | Ga | 0.96 | Partially- | 0.031 Ag + | 0.58 | Synergy | 0.007 Ag + |
| | | | | Synergy | 6.25 Ga | | | 1.56 Ga |
| MHB | Ag | Al | 1 | Partially- | 0.25 Ag + 50 Al | 1 | Partially- | 0.25 Ag + 50 |
| | | | | Synergy | | | Synergy | Al |
| MHB | Ag | Au | 1 | Partially- | 0.065 Ag + | 0.47 | Synergy | 0.007 Ag + |
| | | | | Synergy | 0.125 Au | | | 0.062 Au |
| LB | Ag | Cu | 2 | Antagonistic | 0.065 Ag + | 0.47 | Synergy | 0.015 Ag + |
| | | | | | 8 Cu | | | 1.5 Cu |
| MHB | Ag | Ga | 2 | Antagonistic | 0.031 Ag + | 0.52 | Synergy | 0.007 Ag + |
| | | | | | 2.5 Ga | | | 0.195 Ga |
| MHB | Ag | Cu | 2 | Antagonistic | 0.065 Ag + | 0.7 | Synergy | 0.007 Ag + |
| | | | | | 8 Cu | | | 3 Cu |
| LB | Ag | Se | * | * | * | * | * | * |
| MHB | Ag | Se | * | * | * | <0.75 | Synergy | 0.015 Ag + |
| | | | | | | | | 25 Se |

The fractional biofilm inhibitory concentration (FBIC) <0.8 = Synergy, FBIC >0.8 and ≤1.2 = indifferent, FBIC was >1.2 = antagonistic MHB = Mueller Hinton Broth, LB = Luria-Bertani, SWF = simulated wound fluid. The lowest FBIC (the most effective biofilm inhibition synergism) and FBEC (the most effective biofilm eradication synergism) are highlighted.
* Results could not be determined from the concentration ranges examined experimentally, i.e. the agents did not effectively kill the biofilms.

The combination of Ag with Te was found the most effective anti-biofilm combination against P. aeruginosa with the lowest FBIC (0.093) (the highest level of synergism for biofilm inhibition) and the lowest FBEC (0.24) (the highest synergism for biofilm eradication) in SWF (0.015 mM Ag+0.063 mM TeI) or MHB (0.015 mM Ag+0.008 mM TeI), respectively (Table 3). Yet, many other combinations were also quite effective including, 0.065 mM Ag+4 mM Zn, 0.065 mM Ag+2 mM Cu, 0.03 mM Ag+0.125 mM Au, 0.065 mM Ag+4 mM Ni, 0.125 mM Ag+12.5 mM Se. 0.25 mM Ag+12.5 mM Ga, and 0.25 mM Ag+25 mM Al.

### Example 2: Efficacy Against P. aeruginosa Clinical Isolates

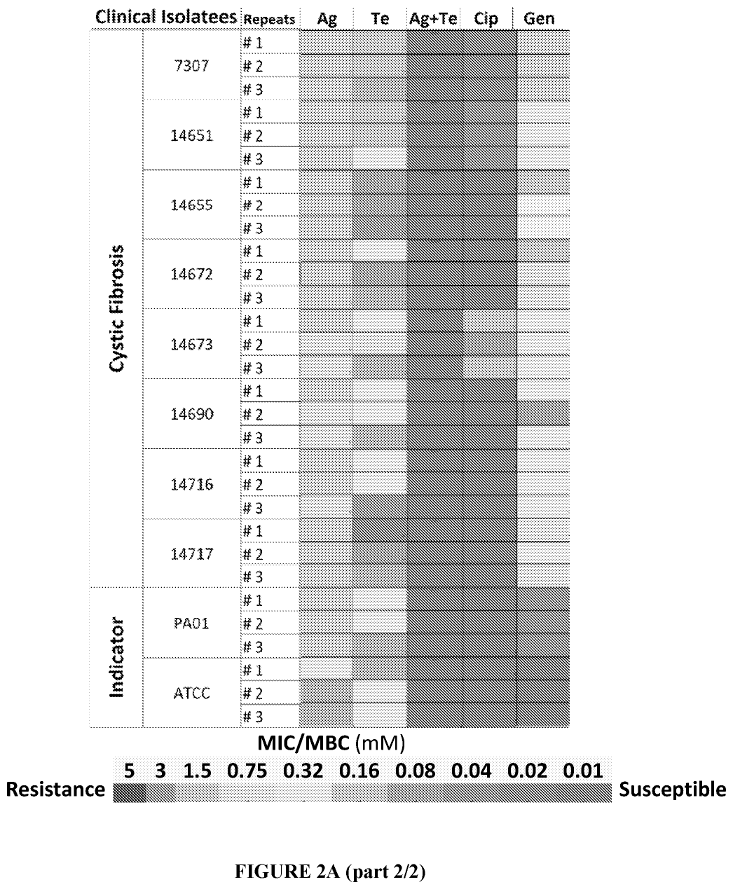

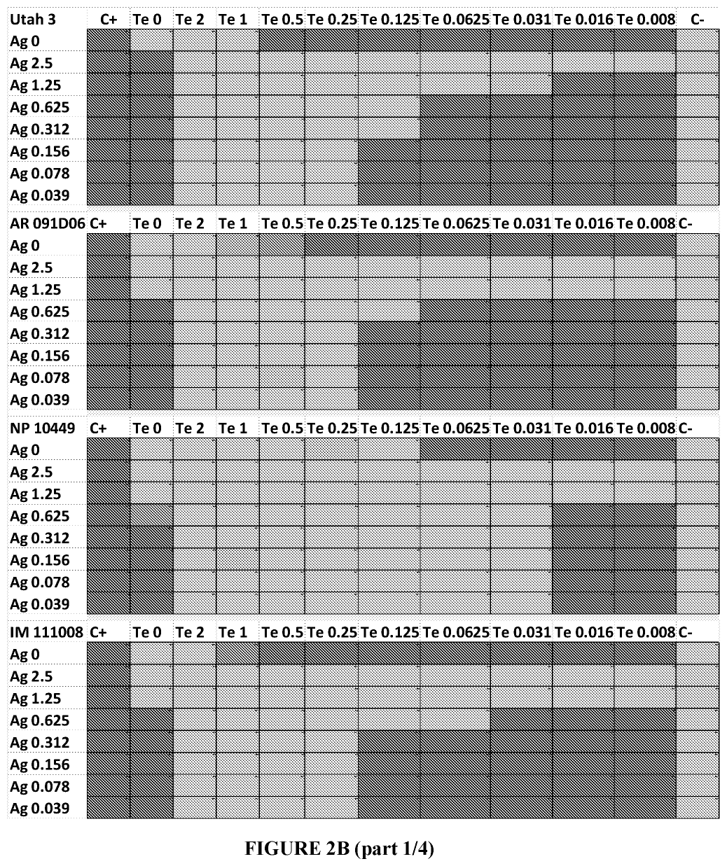

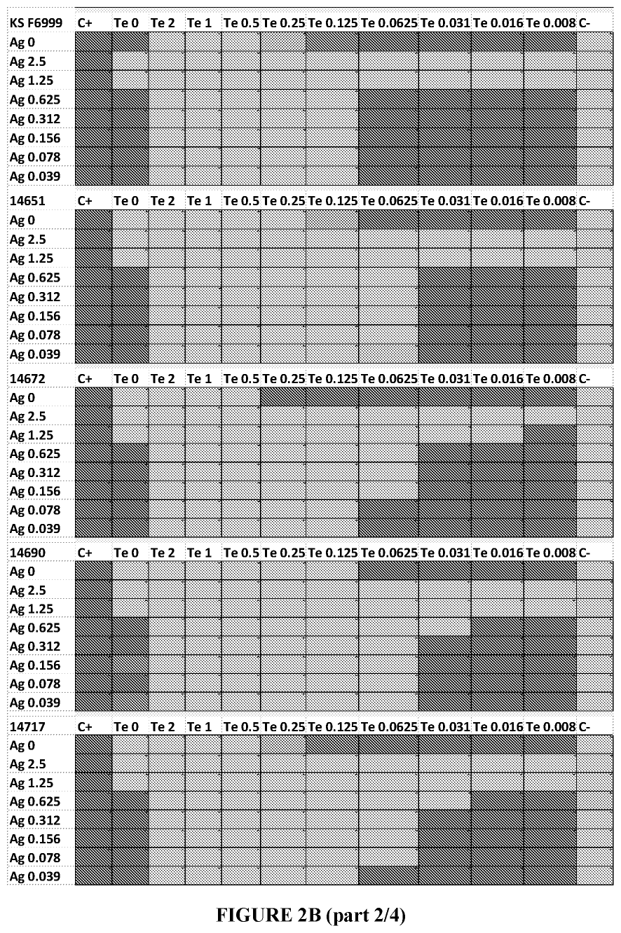

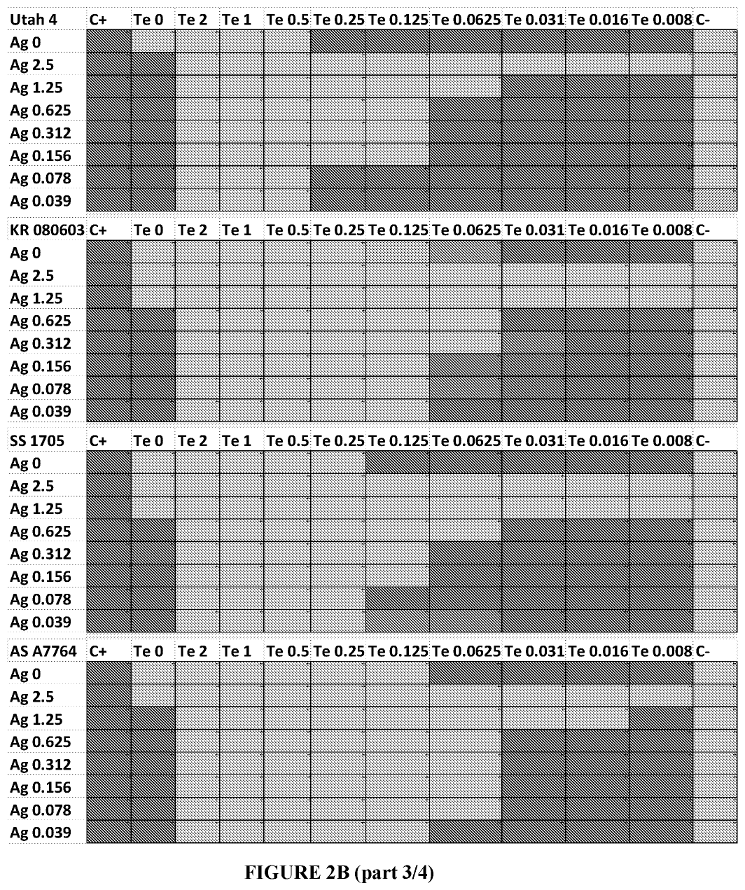

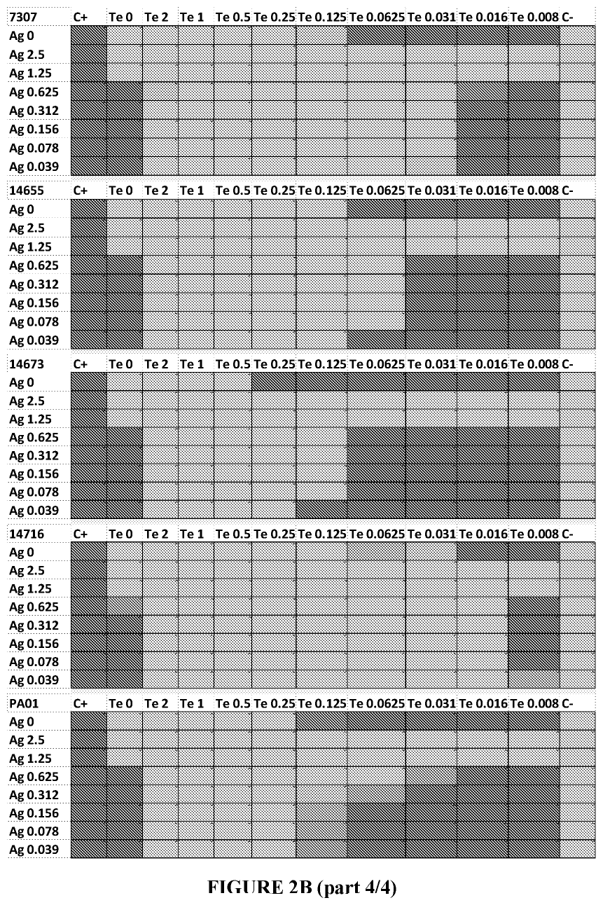

The different MBAs were also tested against clinical isolates to explore the efficacy profiles. As shown in FIGS. 2A, all the clinical antibiotic resistance isolates were highly susceptible to the Ag—Te combination. FIG. 2B illustrated synergism checkboard of synergism biofilm inhibitory of Ag—Te combination on clinical isolates and indicator strains of P. aeruginosa (n=3).

### Example 3: Prevention and Eradication of Staphylococcus aureus Biofilms

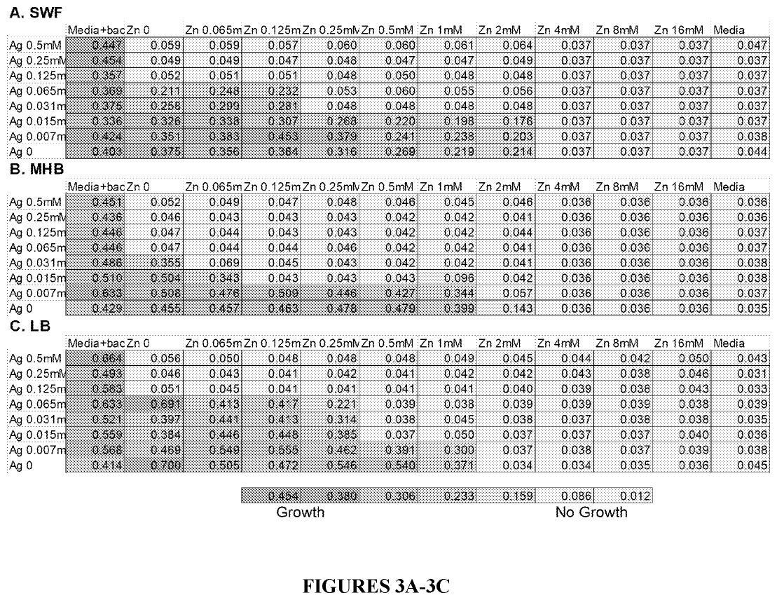

FIG. 3 provides the results of the checkboard of synergism biofilm inhibitory, minimum biofilm inhibition concentration (MBIC) of silver nitrate (Ag), zinc sulfate (Zn), Ag—Zn combination, on indicator strains of Staphylococcus aureus (n=3) in three different media Simulated Wound Fluid (FIG. 3A), MHB (FIG. 3B), and LB (FIG. 3C).

The combination of Ag with Zn was found to be the most effective anti-biofilm combination against S. aureus with the lowest FBIC (0.086) (the highest level of synergism for biofilm inhibition) and the lowest FBEC (0.25) (the highest synergism for biofilm eradication) in SWF (0.031 mM Ag+0.25 mM Zn) and (0.125 mM Ag+1 mM Zn), respectively. Yet, many other combinations are also quite effective including, 0.007 mM Ag+3 mM Te, 0.065 mM Ag+0.125 mM Au, 0.031 mM Ag+2 mM Cu, 0.015 mM Ag+0.125 mM Ni, 0.015 mM Ag+0.1 mM Al, and 0.065 mM Ag+25 mM Se.

TABLE 4
Synergism analysis against Staphylococcus aureus biofilm; estimated fractional biofilm inhibitory
concentration (FBIC) and estimated fractional biofilm eradication concentration (FBEC) values for
combinations of Metal(loid)-Based antimicrobials against Staphylococcus aureus biofilm.

| | Metal(loid)-based | | | | | | | |
|---|---|---|---|---|---|---|---|---|
| | antibiotic | | Biofilm inhibition synergism | | | Biofilm Eradication synergism | | |
| | agent | agent | activity (FBIC) | | | activity (FBEC) | | |
| Media | A | B | FBIC | Interpretation | Conc. (mM) | FBEC | Interpretation | Conc. (mM) |
| SWF | Ag | Zn | 0.086 | Synergy | 0.031 Ag + | 0.25 | Synergy | 0.125 Ag + |
| | | | | | 0.25 Zn | | | 1 Zn |
| MHB | Ag | Zn | 0.24 | Synergy | 0.015 Ag + | 0.3 | Synergy | 0.125 Ag + |
| | | | | | 0.125 Zn | | | 0.5 Zn |
| LB | Ag | Zn | 0.37 | Synergy | 0.015 Ag + | 0.5 | Synergy | 0.125 Ag + |
| | | | | | 0.5 Zn | | | 0.5 Zn |
| SWF | Ag | Te | 0.094 | Synergy | 0.007 Ag + | 0.3 | Synergy | 0.03 Ag + |
| | | | | | 3 Te | | | 6 Te |
| LB | Ag | Au | 1.1 | Partial-synergy | 0.065 Ag + | 0.31 | Synergy | 0.065 Ag + |
| | | | | | 0.125 Au | | | 1 Au |
| LB | Ag | Au | 1.1 | Partial-synergy | 0.065 Ag + | 0.31 | Synergy | 0.065 Ag + |
| | | | | | 0.125 Au | | | 1 Au |
| SWF | Ag | Au | 0.47 | Synergy | 0.007 Ag + | 0.49 | Synergy | 0.031 Ag + |
| | | | | | 0.125 Au | | | 0.25 Au |
| MHB | Ag | Cu | 0.49 | Synergy | 0.031 Ag + | 0.31 | Synergy | 0.031 Ag + |
| | | | | | 2 Cu | | | 4 Cu |
| MHB | Ag | Ni | 0.51 | Synergy | 0.015 Ag + | 1 | Partial-synergy | 0.007 Ag + |
| | | | | | 0.125 Ni | | | 8 Ni |
| MHB | Ag | Te | 0.7 | Synergy | 0.007 Ag + | 1.06 | Partial-synergy | 0.015 Ag + |
| | | | | | 0.06 Te | | | 0.015 Te |
| LB | Ag | Ni | 0.76 | Synergy | 0.125 Ag + | 1 | Partial-synergy | 0.007 Ag + |
| | | | | | 1 Ni | | | 4 Ni |
| SWF | Ag | Ni | 0.97 | Partial-synergy | 0.031 Ag + | 0.37 | Synergy | 0.125 Ag + |
| | | | | | 1 Ni | | | 1 Ni |
| SWF | Ag | Cu | 1 | Partial-synergy | 0.065 Ag + | 0.56 | Synergy | 0.25 Ag + |
| | | | | | 1 Cu | | | 0.5 Cu |
| LB | Ag | Cu | 1 | Partial-synergy | 0.25 Ag + | 0.65 | Synergy | 0.25 Ag + |
| | | | | | 0.5 Cu | | | 1 Cu |
| MHB | Ag | Al | 1 | Partial-synergy | 0.015 Ag + | 1 | Partial-synergy | 0.031 Ag + |
| | | | | | 0.1 Al | | | 0.4 Al |
| SWF | Ag | Al | 1 | Partial-synergy | 0.065 Ag + | 1.25 | antagonistic | 0.007 Ag + |
| | | | | | 12.5 Al | | | 50 Al |
| MHB | Ag | Au | 1.1 | Partial-synergy | 0.015 Ag + | 0.58 | Synergy | 0.065 Ag + |
| | | | | | 0.062 Au | | | 0.016 Au |
| LB | Ag | Al | 1.1 | Partial-synergy | 0.031 Ag + | ND | ND | ND |
| | | | | | 0.8 Al | | | |
| LB | Ag | Te | 1.25 | Antagonistic | 0.065 Ag + | 0.73 | Synergy | 0.25 Ag + |
| | | | | | 0.004 Te | | | 0.06 Te/0.5 Ag + |
| | | | | | | | | 0.03 Te |
| LB | Ag | Se | ND | ND | 0.065 Ag + | ND | ND | 0.125 Ag + |
| | | | | | 25 Se | | | 12.5 Se |
| MHB | Ag | Se | ND | ND | ND | ND | ND | ND |
| SWF | Ag | Se | ND | ND | ND | ND | ND | 0.125 Ag + |
| | | | | | | | | 0.8 Se |

The fractional biofilm inhibitory concentration (FBIC) <0.8 = Synergy, FBIC >0.8 and ≤1.2 = indifferent, FBIC was >1.2 = antagonistic MHB = Mueller Hinton Broth, LB = Luria-Bertani, SWF = simulated wound fluid. The lowest FBIC (the most effective biofilm inhibition synergism) and FBEC (the most effective biofilm eradication synergism) are highlighted.

### Example 4: Prevention of Bacterial Recovery after the Removal of the Challenge

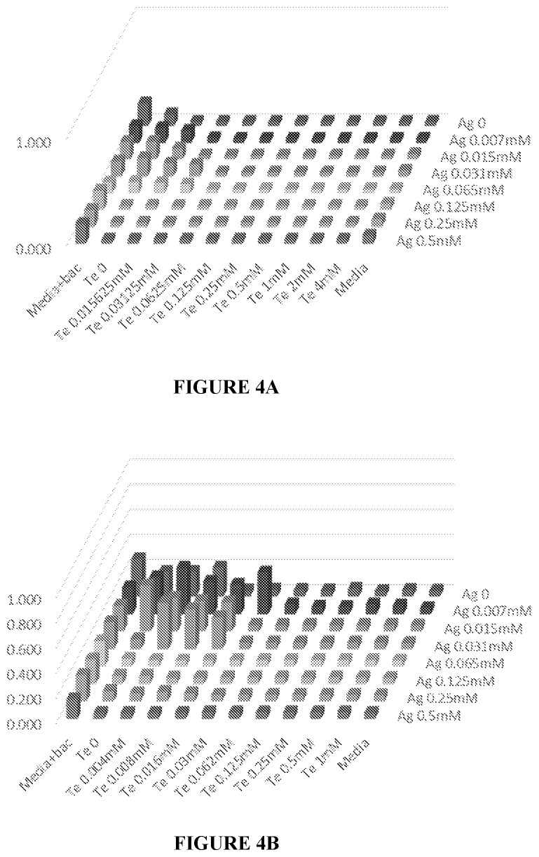

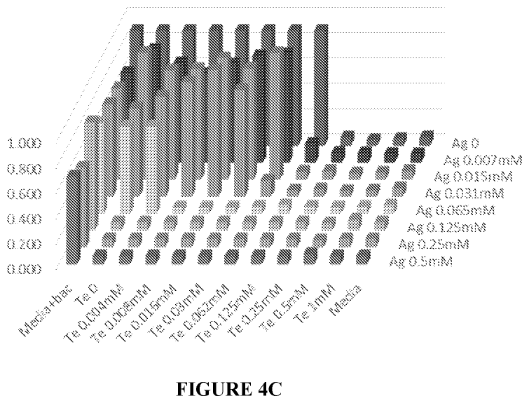

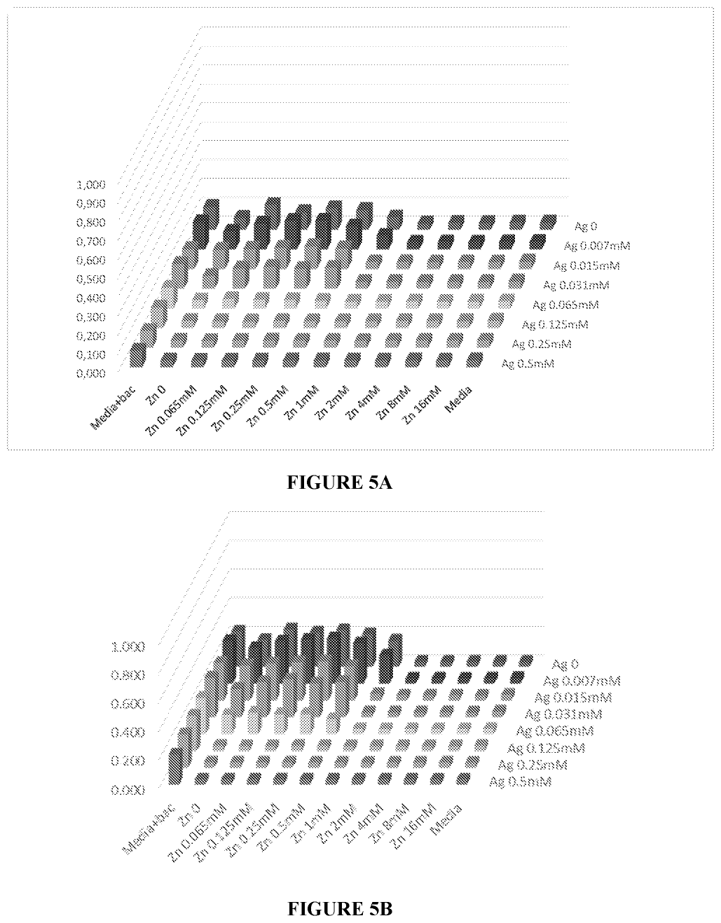

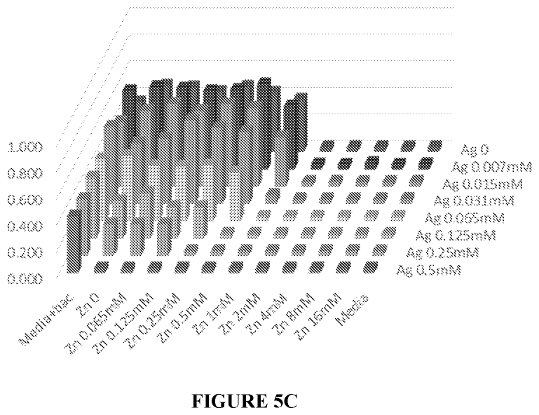

The recovery potency of P. aeruginosa after treatment with Ag—Te combination and each agent individually was tested after 2, 4, and 24 hours. As shown in FIG. 4, the combination of Ag—Te against P. aeruginosa in SWF inhibited the recovery of bacteria in the same fresh media, while the Ag and Te without combination couldn't inhibit the recovery of bacteria and the bacteria recovered in three-fold higher dilution of Ag and two-fold higher dilution of Te when used alone. These results confirm once more the importance of a combination (i.e., here at least one metal and at least one metalloid).

### Example 5: Reduction of Antibiotic Resistance

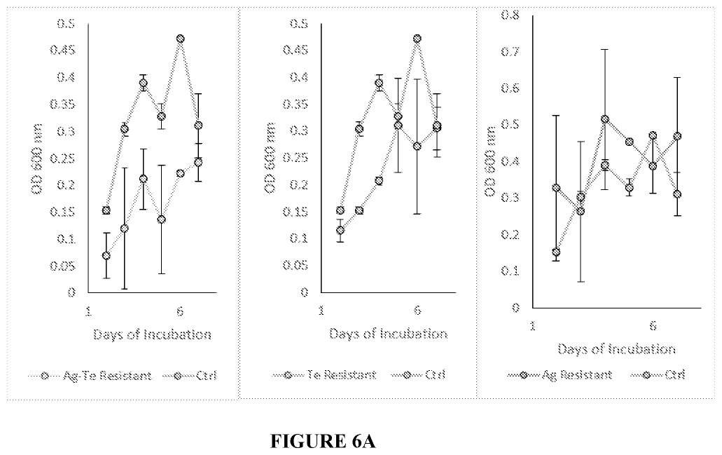

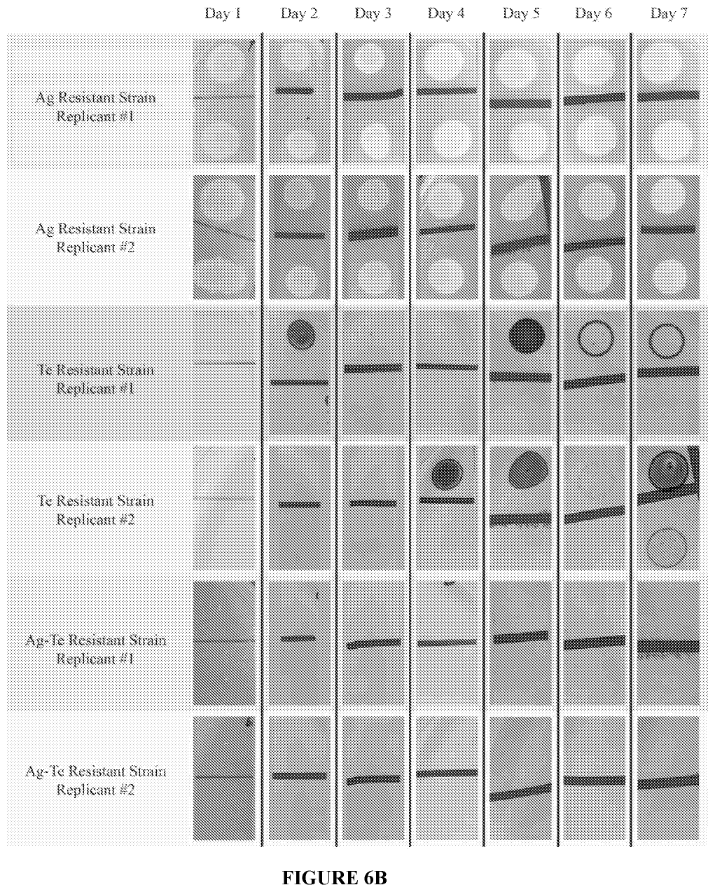

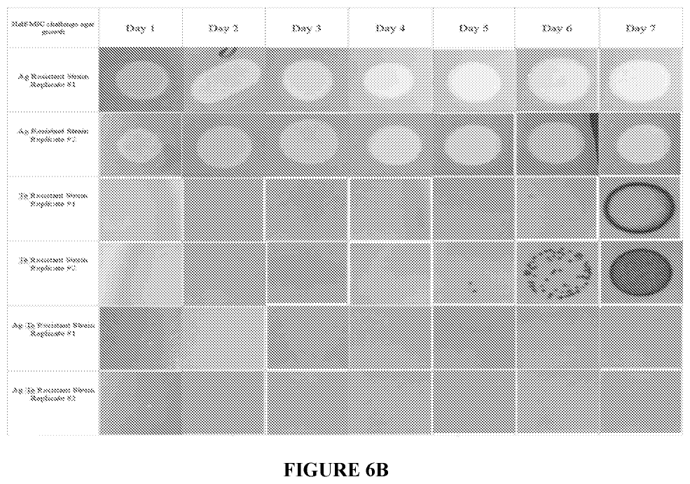

The impact of the MBAs on antibiotic resistance was also tested. Serial passage of growth as a screen for resistant P. aeruginosa PAO1 was performed for 7 days in SWF supplemented with sublethal (1/8 dilution from the MIC) concentrations of Ag, Te, and Ag—Te mixture. Ag resistance occurred after two days, and the tellurite culture obtained resistance after five days of serial passages. As shown in FIGS. 6A and 6B, there was no resistance in Ag/Te combination sublethal concentration within the 7 days of the experiment.

### REFERENCES

1. Michael C A, Dominey-Howes D, Labbate M. The antimicrobial resistance crisis: causes, consequences, and management. Frontiers in public health. 2014; 2:145.
2. Prestinaci F, Pezzotti P, Pantosti A. Antimicrobial resistance: a global multifaceted phenomenon. Pathogens and global health. 2015; 109 (7): 309-18.
3. See the web pages dedicated to Antibiotic/Antimicrobial Resistance (AR/AMR) of the U.S. Centers for Disease Control and Prevention (CDC).
4. Boucher H W, Bakken J S, Murray B E. The United Nations and the urgent need for coordinated global action in the fight against antimicrobial resistance. American College of Physicians; 2016. p. 812-3.
5. Flemming H-C, Wuertz S. Bacteria and archaea on Earth and their abundance in biofilms. Nature Reviews Microbiology. 2019; 17 (4): 247-60.
6. Fratamico P M, Annous B A, Guenther N. Biofilms in the food and beverage industries: Elsevier; 2009.
7. Costerton J W, Stewart P S, Greenberg E P. Bacterial biofilms: a common cause of persistent infections. Science. 1999; 284 (5418): 1318-22.
8. Mohammadi M, Masoumipour F, Hassanshahian M, Jafarinasab T. Study the antibacterial and antibiofilm activity of Carum copticum against antibiotic-resistant bacteria in planktonic and biofilm forms. Microbial Pathogenesis. 2019; 129:99-105.
9. Tenover F C. Mechanisms of antimicrobial resistance in bacteria. The American journal of medicine. 2006; 119 (6): S3-S10.
10. Acar J F. Antibiotic synergy and antagonism. Medical clinics of North America. 2000; 84 (6): 1391-406.
11. Barras F, Aussel L, Ezraty B. Silver and antibiotic, new facts to an old story. Antibiotics. 2018; 7 (3): 79.
12. Sim W, Barnard R T, Blaskovich M, Ziora Z M. Antimicrobial silver in medicinal and consumer applications: a patent review of the past decade (2007-2017). Antibiotics. 2018; 7 (4): 93.
13. Chopra I. The increasing use of silver-based products as antimicrobial agents: a useful development or a cause for concern? Journal of antimicrobial Chemotherapy. 2007; 59 (4): 587-90.
14. Lestari MFWLA. Synergism Effect of Antibiotics and Silver Nanoparticles to Control Antibiotic Resistant Bacteria: A Mini Review. 2021.
15. Pormohammad A, Turner R J. Silver antibacterial synergism activities with eight other metal(loid)-based antimicrobials against Escherichia coli, Pseudomonas aeruginosa, and Staphylococcus aureus. Antibiotics. 2020; 9 (12): 853.
16. Lemire J A, Harrison J J, Turner R J. Antimicrobial activity of metals: mechanisms, molecular targets and applications. Nature Reviews Microbiology. 2013; 11 (6): 371-84.
17. Moormeier D E, Bayles K W. Staphylococcus aureus biofilm: a complex developmental organism. Molecular microbiology. 2017; 104 (3): 365-76.
18. Thi M T T, Wibowo D, Rehm B H. Pseudomonas aeruginosa biofilms. International Journal of Molecular Sciences. 2020; 21 (22): 8671.
19. Mulcahy L R, Isabella V M, Lewis K. Pseudomonas aeruginosa biofilms in disease. Microbial ecology. 2014; 68 (1): 1-12.
20. Lemire J A, Kalan L, Bradu A, Turner R J. Silver oxynitrate, an unexplored silver compound with antimicrobial and antibiofilm activity. Antimicrobial agents and chemotherapy. 2015; 59 (7): 4031-9.

21 Monych N K, Turner R J. Multiple Compounds Secreted by Pseudomonas aeruginosa Increase the Tolerance of Staphylococcus aureus to the Antimicrobial Metals Copper and Silver. mSystems. 2020; 5 (5).
22. Moody J. Synergism testing: broth microdilution checkerboard and broth macrodilution method. Clinical microbiology procedures handbook. 2004:1-28.
23. Harrison J J, Turner R J, Joo D A, Stan M A, Chan C S, Allan N D, et al. Copper and quaternary ammonium cations exert synergistic bactericidal and antibiofilm activity against Pseudomonas aeruginosa. Antimicrobial Agents and Chemotherapy. 2008; 52 (8): 2870-81.
24. Bonapace C R, Bosso J A, Friedrich L V, White R L. Comparison of methods of interpretation of checkerboard synergy testing. Diagnostic microbiology and infectious disease. 2002; 44 (4): 363-6.
25. O'Toole G A. Microtiter dish biofilm formation assay. JoVE (Journal of Visualized Experiments). 2011 (47): e2437.
26. Ceri H, Olson M, Stremick C, Read R, Morck D, Buret A. The Calgary Biofilm Device: new technology for rapid determination of antibiotic susceptibilities of bacterial biofilms. Journal of clinical microbiology. 1999; 37 (6): 1771-6.
27. Harrison J J, Turner R J, Ceri H. High-throughput metal susceptibility testing of microbial biofilms. BMC microbiology. 2005; 5 (1): 1-11.

Headings are included herein for reference and to aid in locating certain sections. These headings are not intended to limit the scope of the concepts described therein, and these concepts may have applicability in other sections throughout the entire specification. Thus, the present invention is not intended to be limited to the embodiments shown herein but is to be accorded the widest scope consistent with the principles and novel features disclosed herein.

The singular forms “a”, “an” and “the” include corresponding plural references unless the context clearly dictates otherwise. Thus, for example, reference to “a metal” includes one or more of such metal and reference to “the method” includes reference to equivalent steps and methods known to those of ordinary skill in the art that could be modified or substituted for the methods described herein.

Unless otherwise indicated, all numbers expressing quantities of ingredients, reaction conditions, concentrations, properties, and so forth used in the specification and claims are to be understood as being modified in all instances by the term “about”. At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contains certain errors resulting from variations in experiments, testing measurements, statistical analyses and such.

It is understood that the examples and embodiments described herein are for illustrative purposes only and that various modifications or changes in light thereof will be suggested to persons skilled in the art and are to be included within the present invention and scope of the appended claims.

1. An anti-biofilm composition comprising a combination of metal(loid) selected from the group consisting of:
a) at least two metals; and
b) at least one metal and at least one metalloid;
wherein said anti-biofilm composition is effective against bacterial biofilm.

2. (canceled)

3. The anti-biofilm composition of claim 1, wherein said combination is a synergistic combination that provides an anti-biofilm activity that is at least 2×, 3×, 4×, 5×, 6×, 7×, 8×, 9×, 10×, 11×, 12×, 13×, 14×, 15×, 16×, 17×, 18×, 19×, 20×, 21×, 22×, 23×, 24×, 25×, 30×, 35×, 40×, 45×, 50×, 75×, 100× or more, greater than the antibiofilm activity of individual metal(loid).

4. (canceled)

5. (canceled)

6. The anti-biofilm composition of any one of claim 1, wherein said anti-biofilm composition is effective against a bacterial biofilm comprising Gram-negative bacteria, and wherein the Gram-negative bacteria is selected from the group consisting of Acinetobacter baumannii, Escherichia coli, Klebsiella pneumoniae, Proteus mirabilis and Pseudomonas aeruginosa.

7. The anti-biofilm composition of claim 1, wherein said anti-biofilm composition is effective for the eradication of bacterial biofilms resulting from the growth of a gram-negative bacteria, and wherein it comprises a combination of silver nitrate and potassium tellurite.

8. (canceled)

9. The anti-biofilm composition of claim 1 wherein said anti-biofilm composition is effective against a bacterial biofilm comprising Gram-positive bacteria, and, wherein the Gram-positive bacteria is selected from the group consisting of Enterococcus faecalis, Enterococcus faecium, Vancomycin-Resistant Enterococcus (VRE), Staphylococcus aureus, Staphylococcus epidermidis, Streptococcus pneumoniae and, Streptococcus pyogenes.

10. The anti-biofilm composition of claim 1, wherein said anti-biofilm composition is effective in the disruption and/or removal of bacterial biofilms resulting from the growth of a gram-positive bacteria, and wherein it comprises a combination of silver nitrate and zinc sulfate.

11. The anti-biofilm composition of claim 1, wherein said anti-biofilm composition is a broad-spectrum composition effective in the eradication of bacterial biofilms resulting from the growth of both gram-positive bacteria and gram-positive bacteria, and wherein it comprises a combination of silver nitrate, potassium tellurite and zinc sulfate.

12. (canceled)

13. The anti-biofilm composition of claim 1, wherein said anti-biofilm composition is effective against at least one of P. aeruginosa ATCC 27853, PAO1 and 39 clinical isolates, as well as Staphylococcus aureus ATCC 25923 strain.

14. The anti-biofilm composition of claim 1, wherein effectiveness against bacterial biofilms comprises at least one of the following activities:
prevention of microbial cell attachment on a surface;
prevention of initiation of biofilm formation;
eradication of a preformed or pre-existing microbial biofilm;
killing microbial cells forming the biofilm;
preventing recovery of microbial cells in a fresh media after exposure with the anti-biofilm composition;
preventing propagation and/or recovery of microbial cells removed from the biofilm;
preventing reformation of a biofilm by bacterial cells previously removed from the biofilm;
effective against clinical antibiotic-resistant strains of microbes;
elimination of persister cells known to be extremely antimicrobial-resistant;
significant decrease in the effective dose which reduces the side effects to the host; and
drastic elimination on the chance of getting resistance in the future.

15. The anti-biofilm composition of claim 1, wherein said anti-biofilm composition provides at least one of the following benefits compared to a control composition comprising only one of said metal and/or metalloid instead of said combination:
higher efficacy at original concentrations;
a reduced effective concentration of said metal and/or metalloid while maintaining antibacterial efficacy;
a decreased host toxicity; and
a decreased likelihood of developing bacterial resistance.

16. The anti-biofilm composition of claim 1, wherein the metal(loid) is selected from the group consisting of silver, copper, gold, zinc, nickel, gallium, aluminium, selenium, tellurium, cobalt, bismuth, thallium, titanium, manganese, zirconium, iron, arsenic, antimonite, cadmium, tin, lead, mercury, vanadium, chromium, tungsten and mixtures thereof.

17. (canceled)

18. The anti-biofilm composition of claim 1, wherein the metal(loid) is selected from the group consisting of silver nitrate (AgNO₃), zinc sulfate (ZnSO₄), nickel sulfate (NiSO₄); copper (II) sulfate, aluminum sulfate (Al₂(SO₄)₃), gallium nitrate (Ga(NO₃)₃, hydrogen tetrachloroaurate (III), potassium tellurite (K₂TeO₃), sodium selenite (Na₂SeO₃), tetrachloroaurate (AuCl₄), nickel sulfate (NiSO₄), and mixtures thereof.

19. The anti-biofilm composition of claim 1, wherein the anti-biofilm composition comprises at least one of silver nitrate, zinc sulfate, and potassium tellurite.

20. The anti-biofilm composition of claim 1, wherein the anti-biofilm composition comprises at least one of the following combinations:
(i) silver nitrate and potassium tellurite;
(ii) silver nitrate and hydrogen tetrachloroaurate (III);
(iii) silver nitrate and nickel sulfate;
(iv) silver nitrate and copper (II) sulfate;
(v) silver nitrate and aluminium sulfate;
(vi) silver nitrate and gallium nitrate;
(vii) silver nitrate and sodium selenite;
(viii) silver nitrate and gallium nitrate; and
(ix) silver nitrate and zinc sulfate.

21. (canceled)

22. The anti-biofilm composition of claim 1, wherein the composition comprises:
0.015 mM Ag+0.063 mM Te;
0.065 mM Ag+0.063 mM Te;
0.5 mM Ag+0.5 mM Te;
0.065 mM Ag+0.065 mM Te;
0.031 mM Ag+0.25 mM Zn;
0.125 mM Ag+1 mM Zn;
0.007 mM Ag+3 mM Te;
0.065 mM Ag+0.125 mM Au;
0.031 mM Ag+2 mM Cu;
0.015 mM Ag+0.125 mM Ni;
0.015 mM Ag+0.1 mM Al; or
0.065 mM Ag+25 mM Se.

23. (canceled)

24. The anti-biofilm composition of claim 1, wherein the metal(loid) is present in the composition as a salt.

25. (canceled)

26. An anti-biofilm composition comprising at least one of the following combinations of metal(loid):
(i) silver nitrate and potassium tellurite;
(ii) silver nitrate and hydrogen tetrachloroaurate (III);
(iii) silver nitrate and nickel sulfate;
(iv) silver nitrate and copper (II) sulfate;
(v) silver nitrate and aluminium sulfate;
(vi) silver nitrate and gallium nitrate;
(vii) silver nitrate and sodium selenite;
(viii) silver nitrate and gallium nitrate; and
(ix) silver nitrate and zinc sulfate.

27. An anti-biofilm composition comprising a combination of silver nitrate and potassium tellurite, and wherein said anti-biofilm composition is effective against bacterial biofilms resulting from the growth of Gram-negative bacteria.

28. (canceled)

29. (canceled)

30. An anti-biofilm composition comprising a combination of silver nitrate and zinc sulfate, wherein said anti-biofilm composition is effective against bacterial biofilms resulting from the growth of Gram-positive bacteria.

31. (canceled)

32. (canceled)

33. A broad-spectrum anti-biofilm composition comprising a combination of silver nitrate, potassium tellurite and zinc sulfate.

34.-50. (canceled)
